(19) United States
(12) Patent Application Publication
(10) Pub. No.: US 2014/0205635 A1
(43) Pub. Date: Jul. 24, 2014

(54) DETECTION OF PRAME GENE EXPRESSION IN CANCER

(71) Applicant: Catherine Minguet, Rixensart (BE)

(72) Inventor: Catherine Minguet, Rixensart (BE)

(73) Assignee: GLAXOSMITHKLINE BIOLOGICAL SA, Rixensart (BE)

(21) Appl. No.: 14/241,548

(22) PCT Filed: Aug. 30, 2012
(86) PCT No.: PCT/EP2012/066920

§ 371 Date: Feb. 27, 2014

(30) Foreign Application Priority Data
Aug. 30, 2011 (GB) 1114919.2

(51) Int. Cl. C12Q 1/68

(52) U.S. Cl. CPC C12Q 1/6886
USPC 424/277.1; 536/24.33; 536/24.31; 435/6.12; 435/6.11

(57) ABSTRACT

The present invention relates to PRAME specific primers and probes, diagnostic kits and methods. The invention further relates to treatment of specific populations of cancer patients suffering from PRAME expressing tumours.

### FIELD OF THE INVENTION

The present invention relates to diagnostic methods and compositions for the detection of PRAME, as well as the immunotherapeutic treatment of populations of patients suffering from PRAME expressing tumours.

### BACKGROUND “PReferentially expressed Antigen in MElanoma”, or “PRAME”, is a tumour antigen encoded by the PRAME gene.

PRAME is an antigen that is over-expressed in many types of tumours, including melanoma, lung cancer and leukaemia (Ikeda et al., Immunity 1997, 6 (2) 199-208). A high level of PRAME expression has been reported for several solid tumors, including ovarian cancer, breast cancer, lung cancer and melanomas, medulloblastoma, sarcomas, head and neck cancers, neuroblastoma, renal cancer, and Wilms' tumour and in hematologic malignancies including acute lymphoblastic and myelogenous leukemias (ALL and AML), chronic myelogenous leukemia (CML), Hodgkin's disease, multiple myeloma, chronic lymphocytic leukemia (CLL) and mantle cell lymphoma (MCL).

PRAME is also expressed at a very low level in a few normal tissues, for example testis, adrenals, ovary and endometrium.

Melanoma

Patients presenting with malignant melanoma in distant metastasis (stage IV according to the American Joint Committee on Cancer (AJCC) classification) have a median survival time of one year, with a long-term survival rate of only 5%. Even the standard chemotherapy for stage IV melanoma has therapeutic response rates of only 8-25%, but with no effect on overall survival. Patients with regional metastases (stage III) have a median survival of two to three years with very low chance of long-term survival, even after an adequate surgical control of the primary and regional metastases. Most patients with stage I to III melanoma have their tumour removed surgically, but these patients maintain a substantial risk for relapse.

Lung Cancer

There are two types of lung cancer: non-small cell lung cancer (NSCLC) and small cell lung cancer (SCLC). The names simply describe the type of cell found in the tumours. NSCLC includes squamous-cell carcinoma, adenocarcinoma, and large-cell carcinoma and accounts for around 80% of lung cancers. NSCLC is hard to cure and treatments available tend to have the aim of prolonging life as far as possible and relieving symptoms of disease. NSCLC is the most common type of lung cancer and is associated with poor outcomes. Of all NSCLC patients, about 25% have loco-regional disease at the time of diagnosis and are still amenable to surgical excision (stages IB, IIA or IIB according to the AJCC classification). However, more than 50% of these patients will relapse within the two years following the complete surgical resection.

PRAME Expression

Primers have been developed for use in real-time PCR assays to determine levels of expression of PRAME in fresh tissue. For example, see Paydas et al., Leukemia Research 31 (2007) 365-369, in which primers for use in detecting PRAME in whole blood have been described:

AF
(SEQ ID NO: 1)
5′-CCA TGA CAA AGA AGC GAA AA-3′
and
AR
(SEQ ID NO: 2)
5′-CAT CTG GCC CAG GTA AGG AG-3′.

Semi-quantitative analyses of the RT-PCR products has also been reported (Proto-Siqueira et al., Leukemia Research 27 (2003) 393-396). In this semi quantitative assay, the following primers were used to determine expression levels of the PRAME gene:

(SEQ ID NO: 3)
5′-CTGTACTCATTTCCAGAGCCAGA-3′
and
(SEQ ID NO: 4)
5′-TATTGAGAGAGGGTTTCCAAGGGGTT-3′.

A difficulty arises with the use of Formalin-Fixed, Paraffin-Embedded (FFPE) tumour tissue, which is the usual method of tumour tissue preservation within clinical centres. The fixation in formalin changes the structure of molecules of RNA within the tissue, causing cross linking and also partial degradation. The partial degradation leads to the creation of smaller pieces of RNA of between 100-300 base pairs. These structural changes to the RNA make it difficult to use RNA extracted from FFPE tissue in conventional diagnostic techniques.

### SUMMARY OF THE INVENTION

Methods and composition s are provided herein to identify tissue in which PRAME gene products, for example nucleic acid such as mRNA, or protein, are expressed.

In one embodiment of the invention there is provided an oligonucleotide comprising, consisting essentially of, or consisting of, the nucleotide sequence of any of SEQ ID NO:5, 6 or 7. In one embodiment of the invention there is provided an oligonucleotide capable of binding under assay conditions to SEQ ID NO:8, 9 or 10 or to the target sequences of SEQ ID NO:5, 6 or 7. The oligonucleotide sequence referred to herein does not include the full length PRAME polynucleotide sequence.

In some embodiments, the oligonucleotide comprises at least six nucleotides of a nucleotide sequence selected from the group consisting of SEQ ID NO:5, 6, 7, 8, 9, and 10. In some embodiments, the at least six nucleotides are the six 3′ nucleotides of SEQ ID NO:5, 6, 7, 8, 9, and 10.

The present disclosure further provides a primer pair comprising SEQ ID NO:5 and/or SEQ ID NO:6.

In a further aspect there is provided a probe comprising the nucleotide sequence of any of SEQ ID NO:5 or SEQ ID NO:7 or the reverse complement of SEQ ID NO:6 (SEQ ID NO:9).

In some embodiments, the probe is chemically modified to prevent extension by a polymerase.

In one embodiment is provided an oligonucleotide set comprising:
(i) a primer pair comprising or consisting of SEQ ID NO:5 and SEQ ID NO:6; and
(ii) a probe comprising or consisting of SEQ ID NO:7.

In one embodiment is provided an oligonucleotide set comprising:
(i) a forward primer comprising or consisting of SEQ ID NO:5
(ii) a reverse primer comprising or consisting of SEQ ID NO:6; and
(iii) a probe comprising or consisting of SEQ ID NO:7.

In a further embodiment of the present invention there is provided a method for determining whether the PRAME gene is expressed in a biological sample, comprising the step of contacting a nucleotide sequence obtained or derived from a biological sample with:
(i) at least one of the oligonucleotides as described herein;
(ii) a set of primers as described herein;
(iii) a probe as described herein; and/or
(iv) an oligonucleotide set as described herein.

In a further embodiment there is provided a method of patient diagnosis comprising the step of contacting a nucleotide sequence obtained or derived from a patient-derived biological sample with one or more of the following components (i) to (iv):
(i) at least one oligonucleotide as described herein;
(ii) a set of primers as described herein;
(iii) a probe as described herein; and/or
(iv) an oligonucleotide set as described herein.

In a further embodiment of the present invention there is provided a method for determining the presence or absence of PRAME positive tumour tissue in a patient-derived biological sample, comprising the step of contacting a nucleotide sequence obtained or derived from a patient-derived biological sample with one or more of the following components (i) to (iv):
(i) at least one oligonucleotide as described herein;
(ii) a set of primers as described herein;
(iii) a probe as described herein; and/or
(iv) an oligonucleotide set as described herein.

In some embodiments of the methods described herein the step of contacting a nucleotide sequence with the one or more of the components (i) to (iv) comprises binding between the nucleotide sequence and one or more of the components (i) to (iv) under assay conditions.

It will be apparent to those skilled in the art that the sequence of the forward primer and probe recognise the sequence of the PRAME nucleic acid and the sequence of the reverse primer recognise the reverse complement of the sequence of the PRAME nucleic acid, and this should be taken into consideration when uses, methods, assays or oligonucleotide sets are developed using the primers and probe sequences disclosed herein.

In embodiments as described herein, the set of primers may amplify a portion (amplicon) of the nucleotide sequence of PRAME and the probe may bind under assay conditions to the nucleotide sequence of the amplicon.

If the primer or probe binds to a nucleic acid derived from a sample, the sample may be identified as expressing the PRAME antigen (PRAME positive). From application of the methods described herein, and/or from analysing the results of the methods described herein, a sample may therefore be identified as PRAME positive tumour tissue.

In one embodiment, the method comprises a step of in situ hybridisation to detect whether the nucleotide sequence binds to the at least one oligonucleotide described herein.

The methods described herein may further comprise a step of determining whether PRAME is expressed in a sample, according to analysis of the results of the methods.

The methods described herein may further comprise a step of determining the presence or absence of PRAME positive tumour tissue according to analysis of the results of the methods.

The methods as described herein may be used on a biological sample which is fresh or which is or has been frozen. Alternatively or additionally, the methods described herein may be performed on a biological sample which is paraffin-preserved, for example Formalin-Fixed, Paraffin-Embedded (FFPE).

Also provided are methods of treating a patient comprising the steps of: determining whether patient-derived tumour tissue expresses the PRAME gene according to a method described herein and then administering a PRAME immunotherapy as described herein to the patient.

In a further embodiment is provided a PRAME immunotherapy for use in the treatment of a patient, in which the patient has been identified as having tissue expressing the PRAME gene (“PRAME-expressing tumour tissue”), using a method described herein.

In one embodiment of the uses or methods of treatment, the patient may have unresected PRAME-expressing tumour tissue (active disease). In a further embodiment, the patient may have had surgical excision of PRAME-expressing tumour tissue (adjuvant setting). In a further embodiment, the patient may first or concurrently receive chemotherapy or radiotherapy to target the tumour tissue.

The present disclosure further provides a method of treating a patient susceptible to recurrence of a PRAME expressing tumour, the patient having been treated to remove/treat PRAME expressing tumour tissue, the method comprising: determining whether the patient's tumour tissue expresses PRAME using a method as described herein and then administering a composition comprising a PRAME specific immunotherapy to said patient.

### BRIEF DESCRIPTION OF THE FIGURES

FIGS. 1-2: Analytical Sensitivity Comparison of AM and GSK RealTime PRAME Oligo Designs.

FIG. 3: A correlation analysis for PRAME Ct results obtained with AM vs. GSK oligo sets.

### DESCRIPTION OF TABLES AND SEQUENCES

Table 1 is a Table showing the sequences of the Forward and Reverse Primers of an embodiment of the present invention.

SEQ ID NO:5 is a contiguous 5′ to 3′ nucleic acid sequence of a Forward Primer for detecting cDNA expression products of PRAME

SEQ ID NO:6 is a contiguous 5′ to 3′ nucleic acid sequence of a Reverse Primer for detecting cDNA expression products of PRAME

SEQ ID NO:7 is a contiguous 5′ to 3′ nucleic acid sequence of a Probe for detecting cDNA expression products of PRAME.

SEQ ID NO:8 is a contiguous 5′ to 3′ nucleic acid sequence of the PRAME gene, recognised by the primer sequence of SEQ ID NO:5 (“Target sequence of SEQ ID NO:5”).

SEQ ID NO:9 is a contiguous 5′ to 3′ nucleic acid sequence of the PRAME gene, recognised by the reverse complement of the primer sequence of SEQ ID NO:6 (“Target sequence of SEQ ID NO:6”).

SEQ ID NO:10 is a contiguous 5′ to 3′ nucleic acid sequence of the PRAME gene, recognised by the probe sequence of SEQ ID NO:7 (“Target sequence of SEQ ID NO:7”).

TABLE 1

| TRAME 2 Taqman set | | Sequence Identifier |
|---|---|---|
| Forward | 5′-GAG-GCC-GCC-TGG-ATC-AG-3′ | SEQ ID NO: 5 |
| Reverse | 5′-CGG-CAG-TTA-GTT-ATT-GAG-AGG-GTT-T-3′ | SEQ ID NO: 6 |
| Probe sequence, | 5′-FAM_TGC-TCA-GGC-ACG-TGA-T_MGB-3′ | SEQ ID NO: 7 |
| showing FAM | | |
| reporter dye, | | |
| nucleotide sequence | | |
| and MGB probe | | |
| Probe nucleotide | TGC-TCA-GGC-ACG-TGA-T | SEQ ID NO: 7 (as |
| sequence only | | above, without the |
| | | dye and probe) |

### DETAILED DESCRIPTION

By biological sample is meant a sample of tissue or cells from a subject that has been removed or isolated from the subject. In some embodiments, the subject is a human patient. By PRAME positive tumour tissue is meant any tissue, for example, tumour tissue or tumour cells, expressing the PRAME gene or the PRAME antigen that has been isolated from a patient.

In one embodiment, the tumour tissue is melanoma; breast cancer; bladder cancer including transitional cell carcinoma; lung cancer including non-small cell lung carcinoma (NSCLC); head and neck cancer including oesophagus carcinoma; squamous cell carcinoma; liver cancer; multiple myeloma and/or colon carcinoma.

In one embodiment, the methods and compositions disclosed herein may be used in the treatment of patients in an adjuvant (post-operative) setting in such cancers particularly lung and melanoma, or in the treatment of metastatic cancers.

In one embodiment, a nucleotide sequence is or has been isolated or purified from a biological sample, for example a tumour tissue sample. In RT-PCR, genomic DNA contamination may lead to false positive results. In one embodiment, genomic DNA is removed or substantially removed from the sample to be tested or included in the methods disclosed herein.

The term “obtained or derived from” as used herein is meant to be used inclusively. That is, it is intended to encompass any nucleotide sequence directly isolated from a tumour sample or any nucleotide sequence derived from the sample for example by use of reverse transcription to produce mRNA or cDNA.

As used herein, the term ‘target sequence’ is a region of the PRAME nucleic acid sequence (either DNA or RNA, e.g. genomic DNA, messenger RNA, or amplified versions thereof) to which the sequence of the probe or primer has partial (i.e. with some degree of mismatch) or total identity; although the reverse primer is the reverse compliment (or, as above, has some degree of mismatch) of the sequence it recognises.

Suitably, the primer or probe may be at least 95% identical to the target sequence over the length of the primer or probe, suitably greater than 95% identical such as 96%, 97%, 98%, 99% and most preferably has 100% identity over its length to the target PRAME sequence. The primers or probes of the invention may be identical to the target sequence at all nucleotide positions of the primer or probe, or may have 1, 2, or more mismatches depending upon the length of probe, temperature, reaction conditions and requirements of the assay, for example. Provided, of course, that the reverse primer fulfils these conditions to the region that is the reverse compliment of the primer sequence.

The term “primer” is used herein to mean any single-stranded oligonucleotide sequence capable of being used as a primer in, for example, PCR technology. Thus, a ‘primer’ according to the invention refers to a single-stranded oligonucleotide sequence that is capable of acting as a point of initiation for synthesis of a primer extension product that is substantially identical (for a forward primer) or substantially the reverse compliment (for a reverse primer) to the nucleic acid strand to be copied. The design (length and specific sequence) of the primer will depend on the nature of the DNA and/or RNA targets and on the conditions at which the primer is used (such as temperature and ionic strength).

The primers may consist of the nucleotide sequences shown in SEQ ID NO:5, 6 or 7, or may consist or comprise of about or exactly 10, 11, 12, 13, 14, 15, 16, 17, 18, 19, 20, 21, 22, 23, 24, 25, 26, 27, 28, 29, 30, 35, 40, 45, 50, 75, 100 or more nucleotides which comprise or fall within the sequences of SEQ ID NO:5, 6 or 7, provided they are suitable for specifically binding a target sequence within a PRAME nucleotide sequence, under assay conditions. When needed, slight modifications of the primers of probes in length or in sequence can be carried out to maintain the specificity and sensitivity required under the given circumstances. Probe and primer sequences of SEQ ID NO:5 to 6 as described herein may be extended or reduced in length by 1, 2, 3, 4, 5 or more nucleotides, for example, in either direction.

In some embodiments, the oligonucleotide comprises at least six nucleotides of a nucleotide sequence selected from the group consisting of SEQ ID NO:5, 6, 7, 8, 9, and 10. In some embodiments, the at least six nucleotides are the six 3′ nucleotides of SEQ ID NO:5, 6, 7, 8, 9, and 10.

“Binding” of a probe to a region of the PRAME nucleotide sequence means that the primer or probe forms a duplex (double-stranded nucleotide sequence) with part of this region or with the entire region under the assay conditions used, and that under those conditions the primer or probe does not form a stable duplex with other regions of the nucleotide sequence present in the sample to be analysed. It should be understood that the primers and probes of the present invention that are designed for specific hybridisation within a region of the PRAME nucleotide sequence may fall entirely within said region or may to a large extent overlap with said region (i.e. form a duplex with nucleotides outside as well as within said region).

In a further aspect of the present invention there is provided a probe comprising the nucleotide sequence of any of SEQ ID NO:5 or SEQ ID NO:7 or the reverse complement of SEQ ID NO:6 (SEQ ID NO:9).

The term “probe” is used herein to mean any single-stranded oligonucleotide sequence capable of binding nucleic acid and being used as a probe in, for example, PCR technology: the probe may consist of the nucleotide sequence shown in SEQ ID NO:7 or may be 5, 6, 7, 8, 9, 10, 11, 12, 13, 14, 15, 16, 17, 18, 19 or 20, 21, 22, 23, 24, 25, 26, 27, 28, 29, 30, 35, 40, 45, 50, 75, 100 or more base pairs which comprise or fall within the sequence of SEQ ID NO:5 or SEQ ID NO:7 or the reverse complement of SEQ ID NO:6 (SEQ ID NO:9) provided they are suitable for specifically binding a target sequence within a PRAME nucleotide sequence.

In one embodiment of the invention, in which a probe is to be used in a method in combination with a pair of primers, the pair of primers should allow for the amplification of part or all of the PRAME polynucleotide fragment to which probes are able to bind or to which the probes are immobilised on a solid support.

The primer and/or probe may additionally comprise a marker, enabling the probe to be detected.

Examples of markers that may be used include: fluorescent markers, for example, 6-carboxyfluorescein (6FAM™), NED™ (Applera Corporation), HEX™ or VIC™ (Applied Biosystems); TET and TAMRA™ markers (Applied Biosystems, CA, USA); chemiluminescent markers, for example Ruthenium probes; and radioactive labels, for example tritium in the form of tritiated thymidine. ³²-Phosphorus may also be used as a radiolabel. Any marker may be used provided that it enables the probe to be detected.

In one embodiment of the present invention, the probe may comprise a fluorescent reporter dye at its 5′-end and a quencher dye at its 3′-end. The fluorescent reporter dye may comprise 6-carboxyfluorescein (6FAM) and the quencher dye may comprise a non-fluorescent quencher (NFQ). Optionally, a Minor Groove Binder protein (MGB™; Applied Biosystems, CA, USA) may be added to the probe, for example the 3′ end of the probe.

In one embodiment, an MGB™ Eclipse Probe may be used (Epoch Biosciences, WA, USA). MGB™ Eclipse probes have an Eclipse™ Dark Quencher and an MGB™ moiety positioned at the 5′-end of the probe. A fluorescent reporter dye is located on the 3′-end of the probe.

In one embodiment, the primer and probe sequences of the present invention may contain or comprise naturally occurring nucleotide structures or bases, for example adenine (A), cytosine (C), guanine (G), thymine (T) and uracil (U). Suitably each nucleotide of the primer or probe can form a hydrogen bond with its counterpart target nucleotide.

Preferably the complementarity of primer or probe with the target sequence is assessed by the degree of A:T and C:G base pairing, such that an adenine (A) nucleotide pairs with a thymine (T), and such that a guanine (G) nucleotide pairs with a cytosine (C), or vice versa. In the RNA form, T may be replaced by U (uracil).

Inosine may be used in universal probes, for example, in which case complementarity may also be assessed by the degree of inosine (probe)—target nucleotide interactions.

In a further embodiment, synthetic or modified analogues of nucleotide structures or bases may be included in the sequence of the probe. By synthetic or modified is meant a non-naturally occurring nucleotide structure or base. Such synthetic or modified bases may replace 1, 2, 3, 4, 5, 6, 7, 8, 9 or all of the bases in the probe sequence. In one embodiment, Cytosine may be replaced by 5-Methyl dC and Thymine may be replaced by 5-Propynyl dU. BHQ2 Quencher may also be included within the sequence.

In one embodiment, an oligonucleotide of the present invention may be used as a probe in a probe based assay. Probe-based assays can be used to exploit oligonucleotide hybridisation to specific sequences and subsequently detect the sequence to which the probe hybridises. Oligonucleotide probes may be labeled using any detection system known in the art. These include, but are not limited to, fluorescent moieties, radioisotope labelled moieties, bioluminescent moieties, luminescent moieties, chemiluminescent moieties, enzymes, substrates, receptors, or ligands.

The oligonucleotide for use as a probe may comprise, consist essentially of, or consist of the nucleotide sequence of any of SEQ ID NO. 5 or 7, or the reverse complement of SEQ ID NO:6 (SEQ ID NO:9).

The primers and probes of the present invention may hybridize directly to nucleic acid or to products of nucleic acid, such as products obtained by amplification. There may also be further purification steps before the amplification product is detected e.g. a precipitation step.

A method of the present invention may further comprise the step of amplifying a nucleotide sequence. In one embodiment, a nucleotide sequence is amplified by Polymerase Chain Reaction (PCR). Alternatively or additionally, a method of the present invention may further comprise contacting an amplified nucleotide sequence with one or more probes as described herein.

The methods of the present invention are suitable for detecting PRAME positive tumour tissue. In one embodiment of the present invention, PRAME positive tissue may be detected using in situ hybridisation. By in situ hybridisation is meant is a hybridisation reaction performed using a primer or probe according to the present invention on intact chromosomes, cells or tissues isolated from a patient for direct visualization of morphologic sites of specific DNA or RNA sequences.

Hybridisation of the polynucleotides may be carried out using any suitable hybridisation method and detection system. Examples of hybridisation systems include conventional dot blot, Southern blots, and sandwich methods. For example, a suitable method may include a reverse hybridisation approach, wherein type-specific probes are immobilised on a solid support in known distinct locations (dots, lines or other figures), and amplified polynucleic acids are labelled in order to detect hybrid formation. The PRAME specific nucleic acid sequences, for example a probe or primer as described herein, can be labelled with biotin and the hybrid can be detected via a biotin-streptavidin coupling with a non-radioactive colour developing system. However, other reverse hybridisation systems may also be employed, for example, as illustrated in Gravitt et al, (Journal of Clinical Microbiology, 1998, 36(10): 3020-3027) the contents of which are also incorporated by reference. Standard hybridisation and wash conditions are described in Kleter et al., Journal of Clinical Microbiology, 1999, 37(8): 2508-2517 and will be optimised under the given circumstances to maintain the specificity and the sensitivity required by the length and sequence of the probe(s) and primer(s).

The methods as described herein may be suitable for use in fresh tissue, frozen tissue, paraffin-preserved tissue and/or ethanol preserved tissue. Well-known extraction and purification procedures are available for the isolation of RNA or DNA from a sample (e.g. in Sambrook et al., 1989). The RNA or DNA may be used directly following extraction from the sample or, more preferably, after a polynucleotide amplification step (e.g. PCR) step. In specific instances, such as for reverse hybridisation assays, it may be necessary to reverse transcribe RNA into cDNA before amplification. In both latter cases the amplified polynucleotide is ‘derived’ from the sample.

The present invention additionally provides a method of treating a patient comprising: determining whether the patient's tumour tissue expresses PRAME using a method as described herein, and administering a PRAME immunotherapy as described herein to said patient. The patient may have tumour tissue expressing PRAME (active disease setting), or may be susceptible to recurrence of a PRAME expressing tumour, the patient having been treated to remove/treat PRAME expressing tumour tissue (adjuvant setting).

The present invention further provides the use of PRAME immunotherapy in the manufacture of a medicament for the treatment of a patient suffering from a PRAME expressing tumour or susceptible to recurrence of a PRAME expressing tumour, in which a patient is identified as having or identified as having had PRAME expressing tumour tissue using a diagnostic method, kit, primer or probe as described herein.

Thus the present invention provides a method for screening, in clinical applications, tissue samples from a human patient for the presence or absence of the expression of PRAME. Such samples could consist of, for example, needle biopsy cores, surgical resection samples or lymph node tissue. For example, these methods include obtaining a biopsy, which is optionally fractionated by crypstat sectioning to enrich tumour cells to about 80% of the total cell population. In certain embodiments, nucleic acids may be extracted from these samples using techniques well known in the art. In other embodiments nucleic acids extracted from the tissue sample may be amplified using techniques well known in the art. The level of PRAME expression can be detected and can be compared with statistically valid groups and/or controls of PRAME negative patients.

In one embodiment, the diagnostic method comprises determining whether a subject expresses the PRAME gene product, for example by detecting the corresponding mRNA and/or protein level of the gene product. For example by using techniques such as Northern blot analysis, reverse transcription-polymerase chain reaction (RT-PCR), semi-quantitative RT-PCR, quantitative RT-PCR, TaqMan PCR, in situ hybridisation, immunoprecipitation, Western blot analysis or immunohistochemistry. According to such a method, cells or tissue may be obtained from a subject and the level of mRNA and/or protein compared to those of tissue not expressing PRAME.

TaqMan PCR Technology

Taq DNA polymerase has 5′-3′ exonuclease activity. The Taqman PCR assay exploits this exonuclease activity to cleave dual-labelled probes annealed to target sequences during PCR amplification.

Briefly, RNA is extracted from a sample and cDNA is synthesised (reverse transcription). The cDNA is then added to a PCR reaction mixture containing standard PCR components (see, for example, components supplied by Roche (CA, USA) for Taqman PCR). The reaction mixture additionally contains a probe that anneals to the template nucleotide sequence between the two primers (ie within the sequence amplified by the PCR reaction, the “amplicon”). The probe comprises a fluorescent reporter dye at the 5′-end and a quencher dye at the 3′-end. The quencher is able to quench the reporter fluorescence, but only when the two dyes are close to each other: this occurs for intact probes.

During and after amplification, the probe is degraded by the Taq DNA polymerase, and any fluorescence is detected.

For quantitative measurements, the PCR cycle number at which fluorescence reaches a threshold value of 10 times the standard deviation of baseline emission is used. This cycle number, called the cycle threshold (Ct), is inversely proportional to the starting amount of target cDNA and allows the amount of cDNA to be measured. Essentially, the more target RNA present in a sample, the lower the Ct number obtained.

The measurements obtained for the Ct value are compared to those obtained for a housekeeping gene. This allows for any errors based on the amount of total RNA added to each reverse transcription reaction (based on wavelength absorbance) and its quality (i.e., degradation): neither of which are reliable parameters to measure the starting material. Therefore, transcripts of a housekeeping gene are quantified as an endogenous control. Beta-actin is one of the most used non-specific housekeeping genes, although others may be used.

Immunotherapy

In one embodiment the PRAME immunotherapy for use in the present invention may be a composition comprising a PRAME antigen, peptide or an epitope thereof (active immunotherapy). In an alternative embodiment, the PRAME immunotherapy may be an antigen binding protein or fragment of an antigen binding protein capable of specifically recognising the PRAME antigen (passive immunotherapy). The antigen binding protein may comprise heavy chain variable regions and light chain variable regions of the invention which may be formatted into the structure of a natural antibody or functional fragment or equivalent thereof.

In one embodiment, the PRAME antigen, peptide or an epitope may be fused or conjugated to a fusion partner or carrier protein. For example, the fusion partner or carrier protein may be selected from protein D, NS1 or CLytA or fragments thereof.

The PRAME protein has 509 amino acids and, in one embodiment, all 509 amino acids of PRAME may be used. However, PRAME constructs with conservative substitutions may also be used. In one embodiment, 1, 2, 3, 4, 5, 6, 7, 8, 9 or more amino acids may be substituted. The PRAME construct may additionally or alternatively contain deletions or insertions within the amino acid sequence when compared to the wild-type PRAME sequence. In one embodiment, 1, 2, 3, 4, 5, 6, 7, 8, 9 or more amino acids may be inserted or deleted.

In one embodiment, the sequence of the PRAME antigen may be 80% or greater than 80%, for example 85%, 90%, 95%, 96%, 97%, 98% or 99% identical to naturally occurring PRAME.

In one aspect the PRAME antigen for use in the present invention comprises a fusion partner protein as described herein and a PRAME antigen or immunogenic fragment thereof.

In one embodiment, the PRAME antigen is a fusion protein comprising:
a) PRAME or an immunogenic fragment thereof, and
b) a heterologous fusion partner derived from protein D,
   wherein the said fusion protein does not include the secretion sequence (signal sequence) of protein D. By secretion or signal sequence or secretion signal of protein D is meant the N-terminal 19 amino acids of protein D. Thus, the fusion partner protein of the present invention may comprise the remaining full length protein D protein, or may comprise approximately the remaining N-terminal third of protein D. For example, the remaining N-terminal third of protein D may comprise approximately or about amino acids 20 to 127 of protein D. In one embodiment, the protein D sequence comprises N-terminal amino acids 20 to 127 of protein D.

The antigen and fusion partner may be chemically conjugated, or may be expressed as a recombinant fusion protein. In an embodiment in which the antigen and partner are expressed as a recombinant fusion protein, this may allow increased levels to be produced in an expression system compared to non-fused protein. Thus the fusion partner may assist in providing T helper epitopes (immunological fusion partner), for example T helper epitopes recognised by humans, and/or the fusion partner may assist in expressing the protein (expression enhancer) at higher yields than the native recombinant protein. In one embodiment, the fusion partner may be both an immunological fusion partner and expression enhancing partner.

In one embodiment of the invention, the immunological fusion partner that may be used is derived from protein D, a surface protein of the gram-negative bacterium, Haemophilus influenza B (WO91/18926) or a derivative thereof. The protein D derivative may comprise the first ⅓ of the protein, or approximately the first ⅓ of the protein. In one embodiment, the first 109 residues of protein D may be used as a fusion partner to provide a PRAME antigen with additional exogenous T-cell epitopes and increase expression level in E. coli (thus acting also as an expression enhancer). In an alternative embodiment, the protein D derivative may comprise the first N-terminal 100-110 amino acids or about or approximately the first N-terminal 100-110 amino acids. In one embodiment, the protein D or derivative thereof may be lipidated and lipoprotein D may be used: the lipid tail may ensure optimal presentation of the antigen to antigen presenting cells

In one embodiment, the PRAME may be Protein D-PRAME/His, a fusion protein comprising from N-terminal to C-terminal: Amino acids Met-Asp-Pro—amino acids 20 to 127 of Protein D-PRAME (509 amino acids or an embodiment as described herein), and optionally a linker and polyhistidine tail (His) may be included that may facilitate the purification of the fusion protein during the production process.

PRAME may be expressed as a fusion protein with protein D at the N terminus and a sequence of seven histidine residues (His tail) at the C-terminus.

In one embodiment of the present invention, the immunotherapy comprises a Protein D-PRAME fusion protein.

A further embodiment of the present invention the immunotherapy comprises a nucleic acid molecule encoding a PRAME specific tumour associated antigen as described herein. In one embodiment of the present invention, the sequences may be inserted into a suitable expression vector and used for DNA/RNA vaccination. Microbial vectors expressing the nucleic acid may also be used as vectored delivered immunotherapeutics.

Examples of suitable viral vectors include retroviral, lentiviral, adenoviral, adeno-associated viral, herpes viral including herpes simplex viral, alpha-viral, pox viral such as Canarypox and vaccinia-viral based systems. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells, yeast or in bacteria may be employed in order to produce quantities of the PRAME protein encoded by the polynucleotides of the present invention, for example for use as subunit vaccines or in immunoassays.

Conventional recombinant techniques for obtaining nucleic acid sequences, and production of expression vectors of are described in Maniatis et al., Molecular Cloning—A Laboratory Manual; Cold Spring Harbor, 1982-1989.

For protein based immunotherapy, the proteins of the present invention are provided either in a liquid form or in a lyophilised form.

Each human dose may comprise 1 to 1000 μg of protein. In one embodiment, the dose may comprise 30-300 μg of protein.

In one embodiment of the present invention the composition comprising a PRAME antigen may further comprise an adjuvant. For example, the adjuvant may comprise one or more or combinations of: 3D-MPL; aluminium salts; CpG containing oligonucleotides; saponin-containing adjuvants such as QS21 or ISCOMs; oil-in-water emulsions; and liposomes. In one embodiment, the adjuvant may comprise 3D-MPL, CpG containing oligonucleotides and QS21, in a liposome formulation.

Suitable vaccine adjuvants for use in the present invention are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF or interleukin-2, -7, or -12, and chemokines, may also be used as adjuvants.

Adjuvants for use in the present invention may comprise a combination of monophosphoryl lipid A, such as 3-de-O-acylated monophosphoryl lipid A (3D-MPL) together with an aluminium salt. 3D-MPL or other toll like receptor 4 (TLR4) ligands such as aminoalkyl glucosaminide phosphates may also be used.

Other known adjuvants that may be used include TLR9 antagonists such as unmethylated CpG containing oligonucleotides. The oligonucleotides are characterised in that the CpG dinucleotide is unmethylated. Such oligonucleotides are well known and are described in, for example WO 96/02555.

The formulation may additionally comprise an oil in water emulsion and/or tocopherol.

Another adjuvant that may be used is a saponin, for example QS21 (Aquila Biopharmaceuticals Inc., Framingham, Mass.), that may be used alone or in combination with other adjuvants. For example, one system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other formulations comprise an oil-in-water emulsion and tocopherol. One adjuvant formulation that may be used in the present invention comprises QS21, 3D-MPL and tocopherol in an oil-in-water emulsion, and is described in WO 95/17210.

In another embodiment, the adjuvants may be formulated in a liposomal composition. The amount of 3 D MPL used is generally small, but depending on the immunotherapy formulation may be in the region of 1-1000 μg per dose, preferably 1-500 μg per dose, and more preferably between 1 to 100 μg per dose.

In one embodiment, the adjuvant may comprise one or more of 3D-MPL, QS21 and an immunostimulatory CpG oligonucleotide. In an embodiment all three immunostimulants are present. In another embodiment 3D MPL and Qs21 are presented in an oil in water emulsion, and in the absence of a CpG oligonucleotide. In one embodiment of the present invention, the adjuvant comprises a CpG oligonucleotide, 3 D -MPL, & QS21 either presented in a liposomal formulation or an oil in water emulsion such as described in WO 95/17210.

The amount of CpG or immunostimulatory oligonucleotides in the adjuvants or immunotherapeutics of the present invention is generally small, but depending on the immunotherapeutic formulation may be in the region of 1-1000 μg per dose, preferably 1-500 μg per dose, and more preferably between 1 to 100 μg per dose.

The amount of saponin for use in the adjuvants of the present invention may be in the region of 1-1000 μg per dose, preferably 1-500 μpg per dose, more preferably 1-250 μg per dose, and most preferably between 1 to 100 μg per dose.

Generally, each human dose may comprise 0.1-1000 μg of antigen, for example 0.1-500 μg, 0.1-100 μg, or 0.1 to 50 μg. An optimal amount for a particular immunotherapy can be ascertained by standard studies involving observation of appropriate immune responses in vaccinated subjects. Following an initial vaccination, subjects may receive one or several booster immunisation adequately spaced.

Other suitable adjuvants include Montanide ISA 720 (Seppic, France), SAF (Chiron, California, United States), ISCOMS (CSL), MF-59 (Chiron), Ribi Detox, RC-529 (GSK, Hamilton, Mont.) and other aminoalkyl glucosaminide 4-phosphates (AGPs).

Throughout this specification and the claims which follow, unless the context requires otherwise, the word “comprise”, and variations such as “comprises” and “comprising”, will be understood to imply the inclusion of a stated integer or step or group of stated integers or steps but not to the exclusion of any other integer or step or group of integers or steps.

The invention will be further described by reference to the following, non-limiting, figures and examples.

### EXAMPLES

### Example 1

### Analytical Sensitivity Comparison of AM and GSK RealTime PRAME Oligo Designs

Purpose

The purpose of this study was to compare the analytical sensitivity of the Abbott Molecular (AM) and GlaxoSmithKline (GSK) oligo designs for the RealTime PRAME assay. To that end, each oligo design was used to test a dilution panel containing a fixed level of beta-Actin RNA and decreasing levels of PRAME RNA. Through this design, panel members with distinct ΔCt values (PRAME Ct minus Actin Ct) will be evaluated.

Method

The GSK oligonucleotide set under evaluation contains one PRAME forward primer (SEQ ID NO:5), one PRAME reverse primer (SEQ ID NO:6) , and one PRAME probe (SEQ ID NO:7), which direct reverse transcription, PCR amplification, and real time fluorescence detection of the PRAME mRNA exon 5/6 region. The GSK oligonucleotide set also contains one beta-Actin forward primer, one beta-Actin reverse primer, and one beta-Actin probe, which direct reverse transcription, PCR amplification, and real time fluorescence detection of the beta-Actin (endogenous control) mRNA exon 5/6 region.

The AM oligonucleotide set under evaluation contains one PRAME forward primer, one PRAME reverse primer, and one PRAME probe, which direct reverse transcription, PCR amplification, and real time fluorescence detection of the PRAME mRNA exon 3/4 region. The AM oligonucleotide The AM oligonucleotide set also contains one beta-Actin forward primer, one beta-Actin reverse primer, and one beta-Actin probe, which direct reverse transcription, PCR amplification, and real time fluorescence detection of the beta-Actin mRNA exon 4/5 region.

To directly compare the performance of the AM and GSK oligo designs, two master mixes were prepared. With the exception of the primers and probes, each master mix contained the same lots of PCR reagents at the same concentrations.

To prepare testing samples for this experiment, PRAME positive RNA from formalin-fixed, paraffin embedded (FFPE) A549 cells was progressively diluted in 30 ng/r×n of RNA from the PRAME mRNA null cell line MDA-MB-231. Five 10-fold serial dilutions of A549 RNA (from 3,000 pg to 0.3 pg/r×n) were generated to achieve a minimal PRAME RNA concentration that produced less than 100% detection. 30 ng/r×n of MDA-MB-231 RNA without A549 RNA was included as a PRAME-negative control.

Each dilution level was tested in replicates of 4 for each master mix on the same m2000rt instrument to assess PRAME and beta-Actin levels.

Linear regressions were calculated from the mean PRAME Ct values relative to the Log₁₀ of A549 concentration (pg/r×n).

Results

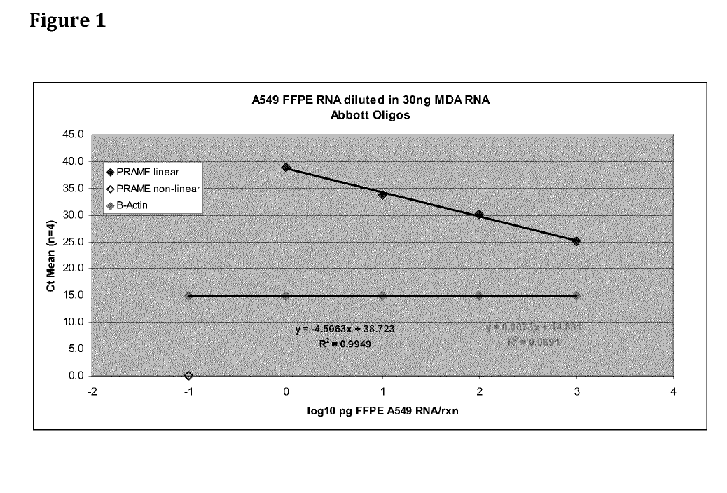

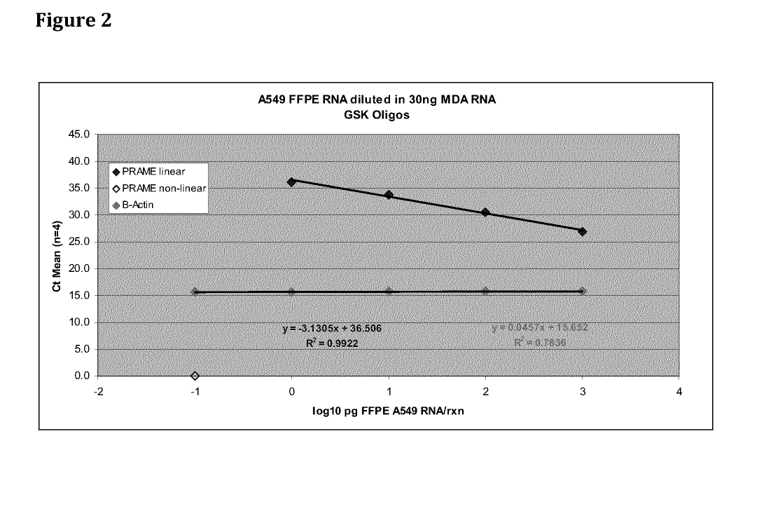

The PRAME detection rate for each dilution panel was similar for both oligo designs, each demonstrating 100% detection (4 of 4 replicates) at 30 pg A549 RNA/r×n and 50% detection (2 of 4 replicates) at 3 pg A549 RNA/r×n. At 0.3 pg A549 RNA/r×n, the GSK design did not detect any PRAME, while the AM design detected PRAME in one replicate. PRAME was not detected in the MDA-MB-231 negative control for either design. PRAME Ct values generated by each oligo set were linear (r²>0.99) across the detectable panel range. Results shown in table 2 and FIGS. 1 and 2.

TABLE 2

| Pg A549 | | Abbott-PRAME | | | | | | GSK-PRAME | | | | | |
|---|---|---|---|---|---|---|---|---|---|---|---|---|---|
| FFPE | Pg MDA | | | | | CT | CT | | | | | CT | CT |
| RNA/rxn | RNA/rxn | CT1 | CT2 | CT3 | CT4 | mean | SD | CT1 | CT2 | CT3 | CT4 | mean | SD |
| 3000 | 30000 | 23.6 | 26.0 | 24.3 | 26.6 | 25.1 | 1.4 | 26.8 | 26.7 | 27.0 | 26.9 | 26.8 | 0.1 |
| 300 | 30000 | 30.3 | 29.9 | 30.2 | 30.2 | 30.1 | 0.2 | 30.4 | 30.6 | 30.5 | 30.3 | 30.4 | 0.2 |
| 30 | 30000 | 33.8 | 34.1 | 33.2 | 33.6 | 33.7 | 0.4 | 34.9 | 33.3 | 33.3 | 33.7 | 33.8 | 0.8 |
| 3 | 30000 | 39.2 | n/a | 38.7 | n/a | 38.9 | 0.4 | n/a | n/a | 36.1 | 36.2 | 36.2 | 0.1 |
| 0.3 | 30000 | n/a | 50.8 | n/a | n/a | 50.8 | n/a | n/a | n/a | n/a | n/a | n/a | n/a |
| 0 | 30000 | n/a | n/a | n/a | n/a | n/a | n/a | n/a | n/a | n/a | n/a | n/a | n/a |

Conclusions

At the highest dilutions, the Ct of samples for which PRAME is detected are lower for the GSK primers than the Abbott primers. The sensitivity of the GSK primer set is therefore higher on the cell line RNA than that of the AM primer set.

The theoretical slope of the regression of Ct versus log10(concentration) for a PCR reaction with 100% efficiency is −3.322. The efficiency (in %) of a PCR reaction can be calculated using the following equaiton: Eff %=((10̂(−1/slope))−1)*100. The slope of the regression line for the Abbott primers is −4.5063, which corresponds to a PCR efficiency of 66.7% (FIG. 1). For the GSK primers, the slope is −3.13, which corresponds to a PCR efficiency of 108.7% (FIG. 2). The efficiency of the GSK primers is closer to the theoretical efficiency than the efficiency of the Abbott primers. It is also generally accepted that PCR reactions with efficiencies below 90% should be redesigned (e.g. http://www.dorak.info/genetics/glosrt.html).

### Example 2

### Comparison of Abbott and GSK Oligos Using 7 FFPE NSCLC Samples

Purpose

The purpose of this experiment was to compare the performance of the Abbott Molecular (AM) and GlaxoSmithKline (GSK) oligo designs for the RealTime PRAME assay. To that end, each oligo design was used to test RNA eluates from seven non-macrodissected Formalin-fixed, Paraffin-embedded (FFPE) Non-small Lung Cancer (NSCLC) specimens.

Method

The GSK oligonucleotide set under evaluation contains one PRAME forward primer, one PRAME reverse primer, and one PRAME probe, which direct reverse transcription, PCR amplification, and real time fluorescence detection of the PRAME mRNA exon 5/6 region. The GSK oligonucleotide set also contains one beta-Actin forward primer, one beta-Actin reverse primer, and one beta-Actin probe, which direct reverse transcription, PCR amplification, and real time fluorescence detection of the beta-Actin (endogenous control) mRNA exon 5/6 region.

The AM oligonucleotide set under evaluation contains one PRAME forward primer, one PRAME reverse primer, and one PRAME probe, which direct reverse transcription, PCR amplification, and real time fluorescence detection of the PRAME mRNA exon 3/4 region. The AM oligonucleotide set also contains one beta-Actin forward primer, one beta-Actin reverse primer, and one beta-Actin probe, which direct reverse transcription, PCR amplification, and real time fluorescence detection of the beta-Actin mRNA exon 4/5 region.

To directly compare real time PCR performance of the AM and GSK oligo designs, two master mixes were prepared. With the exception of the primers and probes, each master mix contained the same lots of PCR reagents at the same concentrations.

To prepare testing samples for this experiment, tissue sections from each of seven FFPE NSCLC specimens were deparaffinized and stained with Nuclear Fast Red. RNA was then extracted from the whole-tissue sections (non-macrodissected) and quantitated using a Nanodrop spectrophotometer.

10 ng of RNA from each specimen was tested in replicates of three for each master mix on an m2000rt instrument to assess PRAME and beta-Actin levels.

Results

For each PCR replicate tested, the PRAME cycle threshold (Ct) and the ΔCt (PRAME Ct minus beta-Actin Ct) obtained with AM and GSK oligo sets are shown in tables 3 and 4. For 3 of the 7 samples tested, PRAME signal (Ct) was undetectable with either oligo set. Of the 4 samples that yielded detectable PRAME signal, one had all 3 replicates detected for either oligo set; one had one replicate detected by AM oligo and 3 replicates detected by GSK oligo; and 2 had 2 replicates detected by AM oligo and one replicate detected by GSK oligo.

TABLE 3

| Sam- | | Abbott-PRAME | | | | GSK-PRAME | | | |
|---|---|---|---|---|---|---|---|---|---|
| ple | Sample | | | | CT | | | | CT |
| ID | input | CT1 | CT2 | CT3 | mean | CT1 | CT2 | CT3 | mean |
| 1 | 10 ng | −1 | −1 | 36.97 | 36.97 | 37 | 35.95 | 35 | 35.98 |
| 2 | 10 ng | 32.02 | 32.55 | 33.53 | 32.7 | 33.02 | 33.35 | 34.03 | 33.47 |
| 3 | 10 ng | −1 | −1 | −1 | −1 | −1 | −1 | −1 | −1 |
| 4 | 10 ng | −1 | −1 | −1 | −1 | −1 | −1 | −1 | −1 |
| 5 | 10 ng | −1 | 37.51 | 37.4 | 37.46 | −1 | 35.82 | −1 | 35.82 |
| 8 | 10 ng | −1 | −1 | −1 | −1 | −1 | −1 | −1 | −1 |
| 9 | 10 ng | −1 | 38.33 | 34.73 | 36.53 | −1 | 36.11 | −1 | 36.11 |

TABLE 4

| Sam- | | Abbott-PRAME | | | | GSK-PRAME | | | |
|---|---|---|---|---|---|---|---|---|---|
| ple | Sample | | | | ΔCT | | | | ΔCT |
| ID | input | ΔCT1 | ΔCT2 | ΔCT3 | mean | ΔCT1 | ΔCT2 | ΔCT3 | mean |
| 1 | 10 ng | n/a | n/a | 14.31 | 14.31 | 13.78 | 12.67 | 11.80 | 12.75 |
| 2 | 10 ng | 9.76 | 10.33 | 11.30 | 10.46 | 10.11 | 10.42 | 11.16 | 10.56 |
| 3 | 10 ng | n/a | n/a | n/a | n/a | n/a | n/a | n/a | n/a |
| 4 | 10 ng | n/a | n/a | n/a | n/a | n/a | n/a | n/a | n/a |
| 5 | 10 ng | n/a | 16.32 | 16.23 | 16.28 | n/a | 14.15 | n/a | 14.17 |
| 8 | 10 ng | n/a | n/a | n/a | n/a | n/a | n/a | n/a | n/a |
| 9 | 10 ng | n/a | 15.36 | 11.90 | 13.66 | n/a | 12.66 | n/a | 12.71 |

Conclusions

At the highest dilutions the GSK oligos detect the PRAME target at lower Ct and lower delta Ct values than the AM assay. Thus, the sensitivity of the GSK assay on the clinical samples is better than that of the AM assay.

### Example 3

### Comparison of Abbott and GSK Oligos Using 50 FFPE NSCLC Samples

Purpose

The purpose of this study was to compare the performance of the Abbott Molecular (AM) and GlaxoSmithKline (GSK) oligo designs for the RealTime PRAME assay. To that end, each oligo design was used to test RNA eluates from 50 macrodissected Formalin-fixed, Paraffin-embedded (FFPE) Non-small Lung Cancer (NSCLC) specimens.

Method

The GSK oligonucleotide set under evaluation contains one PRAME forward primer, one PRAME reverse primer, and one PRAME probe, which direct reverse transcription, PCR amplification, and real time fluorescence detection of the PRAME mRNA exon 5/6 region.

The AM oligonucleotide set under evaluation contains one PRAME forward primer, one PRAME reverse primer, and one PRAME probe, which direct reverse transcription, PCR amplification, and real time fluorescence detection of the PRAME mRNA exon 3/4 region.

In this study, both oligonucleotide sets also contain one beta-Actin forward primer, one beta-Actin reverse primer, and one beta-Actin probe, which direct reverse transcription, PCR amplification, and real time fluorescence detection of the beta-Actin (endogenous control) mRNA exon 5/6 region.

To directly compare real time PCR performance of the AM and GSK oligo designs, two master mixes were prepared. With the exception of the PRAME primers and probes, each master mix contained the same lots of PCR reagents at the same concentrations.

To prepare testing samples for this experiment, tissue sections from each of 50 FFPE NSCLC specimens were deparaffinized, stained with Nuclear Fast Red, and macrodissected to achieve a minimum of 50% tumor cells in an area of at least 50 mm². RNA was then extracted from each macrodissected specimen and quantitated using a Nanodrop spectrophotometer.

50 ng of RNA was tested in replicates of 2 for each master mix on an m2000rt instrument to assess PRAME and beta-Actin levels, except for two specimens for which only one replicate was tested due to lack of sufficient sample and for one specimen for which 25 ng was tested in each of the two replicates due to lack of sufficient sample. Due to space limitations on the PCR plate, specimens were tested in two separate batches for each master mix.

Results

For 48 out of the 50 specimens, the AM oligo set detected PRAME signal (Ct) in 100% of the tested PCR replicates. For the remaining two specimens, the AM oligo set detected PRAME signal in one of the two PCR replicates. The AM oligo set detected beta-Actin signal in all PCR replicates tested for all specimens. The GSK oligo set detected PRAME signal and beta-Actin signal in all PCR replicates tested for all specimens.

For each specimen tested, the mean PRAME cycle threshold (Ct), the mean beta-Actin Ct, and the ΔCt (mean PRAME Ct minus mean beta-Actin Ct) obtained with AM and GSK oligo sets are shown in tables 5 and 6.

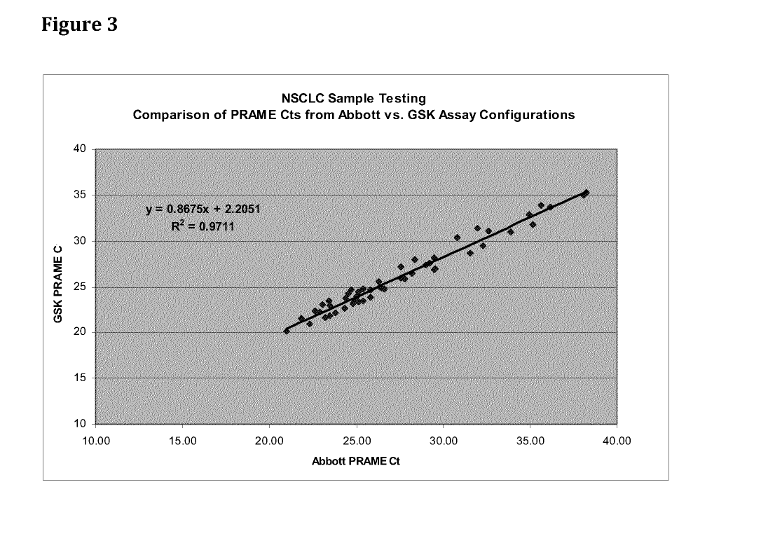

A correlation analysis for PRAME Ct results obtained with AM vs. GSK oligo sets is shown in FIG. 3.

TABLE 5

| Patient | | Abbot Mastermix | | | GSK Mastermix | | |
|---|---|---|---|---|---|---|---|
| ID | RNA ID | PRAME_CY5_Ct | ACTIN_FAM_Ct | D ct | PRAME_CY5_Ct | ACTIN_FAM_Ct | D ct |
| 31 | 93197 | 26.48 | 15.05 | 11.43 | 24.83 | 14.99 | 9.84 |
| 32 | 93264 | 24.92 | 13.85 | 11.07 | 23.56 | 14.09 | 9.47 |
| 33 | 93451 | 35.18 | 15.07 | 20.11 | 31.82 | 15.03 | 16.80 |
| 42 | 83286 | 23.20 | 14.23 | 8.97 | 21.65 | 14.25 | 7.40 |
| 43 | 83287 | 21.00 | 14.959 | 6.05 | 20.12 | 14.86 | 5.26 |
| 71 | 83060 | 29.51 | 16.38 | 13.13 | 26.89 | 16.15 | 10.74 |
| 73 | 87858 | 23.81 | 14.26 | 9.55 | 22.10 | 14.33 | 7.77 |
| 74 | 90724 | 25.14 | 15.29 | 9.86 | 24.49 | 15.28 | 9.21 |
| 89 | 91970 | 34.96 | 16.25 | 18.72 | 32.85 | 16.09 | 16.77 |
| 95 | 82840 | 25.83 | 15.63 | 10.20 | 23.86 | 15.46 | 8.40 |
| 98 | 87020 | 24.81 | 14.91 | 9.90 | 23.10 | 14.78 | 8.32 |
| 101 | 89659 | 23.44 | 13.14 | 10.30 | 23.43 | 13.39 | 10.04 |
| 135 | 100205 | #32.3 | 19.61 | 12.69 | #29.44 | 19.45 | #9.995 |
| 141 | 92803 | 35.67 | 15.40 | 20.27 | 33.91 | 15.35 | 18.56 |
| 252 | 83226 | 22.65 | 14.46 | 8.19 | 22.38 | 14.40 | 97.98 |
| 253 | 83457 | 24.56 | 15.57 | 9.00 | 24.24 | 15.61 | 8.63 |
| 255 | 91148 | 27.58 | 16.01 | 11.58 | 25.92 | 15.93 | 9.99 |
| 256 | 89167 | 22.34 | 14.44 | 7.90 | 20.90 | 14.44 | 6.46 |
| 258 | 91820 | 38.26(1) | 15.65 | 22.61 | 35.33 | 15.40 | 19.94 |
| 259 | 95217 | 32.64 | 14.61 | 18.03 | 31.07 | 14.40 | 16.68 |
| | water | NA | NA | NA | NA | NA | NA |

(n) = Number in parentheses indicates reps detected (out of 2) when less than 100% detection
#25 ng sample/well was used because of lack of enough sample

TABLE 6

| Patient | | Abbot Mastermix | | | GSK Mastermix | | |
|---|---|---|---|---|---|---|---|
| ID | RNA ID | PRAME_CY5_Ct | ACTIN_FAM_Ct | D ct | PRAME_CY5_Ct | ACTIN_FAM_Ct | D ct |
| 260 | 98823 | 25.42 | 14.34 | 11.12 | 23.48 | 14.06 | 9.42 |
| 261 | 98833 | 23.07 | 15.06 | 8.13 | 23.04 | 15.03 | 8.01 |
| 262 | 99835 | 30.82 | 14.64 | 16.29 | 30.37 | 14.65 | 15.72 |
| 305 | 84520 | 36.16 | 16.52 | 19.69 | 33.68 | 16.42 | 17.26 |
| 313 | 100332 | *26.33 | *17.71 | *8.62 | *25.00 | *17.45 | *7.55 |
| 357 | 95347 | 22.88 | 14.39 | 8.61 | 22.27 | 14.21 | 8.06 |
| 456 | 82315 | 29.51 | 17.51 | 12.03 | 28.15 | 17.26 | 10.89 |
| 523 | 91128 | 29.56 | 17.10 | 12.51 | 26.98 | 16.82 | 10.16 |
| 601 | 85029 | 25.39 | 15.47 | 10.04 | 24.79 | 15.21 | 9.58 |
| 602 | 87515 | 38.11(1) | 17.77 | 20.47 | 34.95 | 17.41 | 17.54 |
| 605 | 89024 | 28.23 | 15.03 | 13.26 | 26.44 | 14.91 | 11.54 |
| 608 | 93474 | 27.80 | 16.42 | 11.33 | 25.83 | 16.44 | 9.40 |
| 633 | 87017 | 29.01 | 16.36 | 12.58 | 27.41 | 16.36 | 11.05 |
| 636 | 92375 | 31.55 | 17.98 | 13.55 | 28.70 | 17.80 | 10.91 |
| 661 | 85031 | 25.01 | 14.58 | 10.46 | 23.96 | 14.60 | 9.37 |
| 662 | 85030 | 25.14 | 13.70 | 11.52 | 23.35 | 13.66 | 9.69 |
| 664 | 87300 | 26.30 | 15.32 | 10.91 | 25.57 | 15.55 | 10.02 |
| 665 | 87608 | 23.49 | 14.31 | 9.10 | 21.85 | 14.41 | 7.44 |
| 697 | 92554 | 27.57 | 17.38 | 10.18 | 27.21 | 17.37 | 9.84 |
| 699 | 100595 | 25.82 | 18.80 | 6.96 | 24.69 | 18.61 | 6.08 |
| 798 | 87505 | 24.41 | 14.34 | 9.97 | 23.71 | 14.40 | 9.31 |
| 800 | 88347 | 24.33 | 14.88 | 9.36 | 22.65 | 14.95 | 7.70 |
| 809 | 94956 | 33.88 | 16.30 | 17.57 | 31.01 | 16.19 | 14.82 |
| 834 | 100357 | 28.35 | 15.06 | 12.99 | 27.95 | 15.22 | 12.73 |
| 852 | 93781 | *29.24 | *19.18 | *10.03 | *21.59 | *18.87 | *8.72 |
| 1203 | 85757 | 24.71 | 14.41 | 10.33 | 24.64 | 14.58 | 10.06 |
| 1205 | 86473 | 26.60 | 14.53 | 12.10 | 24.70 | 14.50 | 10.20 |
| 1207 | 87510 | 32.00 | 14.79 | 17.31 | 31.38 | 14.70 | 16.68 |
| 1212 | 93262 | 21.86 | 14.29 | 7.60 | 21.54 | 14.34 | 7.20 |
| 1216 | 101054 | 23.50 | 14.04 | 9.44 | 22.90 | 14.20 | 8.70 |
| | Water | NA | NA | NA | NA | NA | NA |

(n) = Number in parentheses indicates reps detected (out of 2) when less than 100% detection
*only one rep was tested because of lack of enough sample

Conclusions

The detection rate of PRAME in the clinical samples is 96% for the AM assay and 100% for the GSK assay.

The intercept of the regression of GSK Ct values versus AM Ct values is 2.2. Therefore, detection of PRAME in the clinical samples occurs on average 2.2 Ct earlier for the GSK assay than for the AM assay. This confirms the better sensitivity of the GSK assay on clinical samples.

The slope of the regression of GSK Ct values versus AM Ct values is 0.8675. Equivalence of the GSK and PRAME assays would result in a slope of 1. This means that the Ct values for the AM assay increase more rapidly (13.3%) than those of the GSK assay as the PRAME concentration decreases. This also highlights better performance of the GSK primers.

1. An oligonucleotide comprising a nucleotide sequence selected from the group consisting of SEQ ID NO:5 to 7.

2-5. (canceled)

6. A kit comprising:
(i) a forward primer comprising SEQ ID NO:5
(ii) a reverse primer comprising SEQ ID NO:6; and
(iii) a probe comprising SEQ ID NO:7.

7-8. (canceled)

9. A method for determining the presence or absence of PRAME positive tumour tissue in a patient-derived biological sample, comprising the step of contacting a nucleotide sequence obtained or derived from a patient-derived biological sample with one or more components selected from the group consisting of:
(i) at least one oligonucleotide of claim 1;
(ii) a set of primers comprising an oligonucleotide comprising SEQ ID NO:5 and an oligonucleotide comprising SEQ ID NO:6;
(iii) a probe comprising SEQ ID NO:7; and/or
(iv) a forward primer comprising SEQ ID NO:5, a reverse primer comprising SEQ ID NO:6; and a probe comprising SEQ ID NO:7.

10. The method of claim 9, further comprising the step of amplifying a nucleotide sequence and detecting in the sample the amplified nucleotide sequence.

11. The method of claim 10, further comprising the step of detecting whether the nucleotide sequence hybridises to component (iii).

12. The method of claim 11 in which the biological sample is Formalin-Fixed, Paraffin-Embedded tissue.

13. A method of treating a patient comprising using the method of claim 9 to select a patient having PRAME positive tumour tissue and then administering a PRAME immunotherapy to the patient.

14-19. (canceled)


SEQUENCE LISTING (7 sequences)

SEQ ID NO: 1 - DNA, 20 bp, Artificial Sequence
  PCR primer
ccatgacaaagaagcgaaaa

SEQ ID NO: 2 - DNA, 20 bp, Artificial Sequence
  PCR primer
catctggcccaggtaaggag

SEQ ID NO: 3 - DNA, 23 bp, Artificial Sequence
  PCR primer
ctgtactcatttccagagccaga

SEQ ID NO: 4 - DNA, 26 bp, Artificial Sequence
  PCR primer
tattgagagagggtttccaaggggtt

SEQ ID NO: 5 - DNA, 17 bp, Artificial Sequence
  PCR primer
gaggccgcctggatcag

SEQ ID NO: 6 - DNA, 25 bp, Artificial Sequence
  PCR primer
cggcagttagttattgagagggttt

SEQ ID NO: 7 - DNA, 16 bp, Artificial Sequence
  PRAME probe
tgctcaggcacgtgat